(19) United States
(12) Patent Application Publication
(10) Pub. No.: US 2012/0133888 A1
(43) Pub. Date: May 31, 2012

(54) SCANNING OPHTHALMOSCOPES

(75) Inventors: Daniel Curtis Gray, Dunfermline (GB); Robert Wall, Penicuik (GB); Graig Robertson, Aberdour (GB); David Cairms, Kinross (GB)

(73) Assignee: OPTOS PLC, Dunfermline, Fife (GB)

(21) Appl. No.: 13/318,018

(22) PCT Filed: Apr. 30, 2010
(86) PCT No.: PCT/GB2010/050713

§ 371 Date: Feb. 8, 2012

(30) Foreign Application Priority Data
May 1, 2009 (GB) 0907557.3

(51) Int. Cl. A61B 3/12; A61B 3/14

(52) U.S. Cl. 351/206; 351/221; 351/246

(57) ABSTRACT

The invention provides a scanning ophthalmoscope for scanning the retina of an eye and method of operating the same. The scanning ophthalmoscope comprises a source of collimated light, a first scanning element and a second scanning element. The source of collimated light and the first and second scanning elements combine to provide a two-dimensional collimated light scan from an apparent point source. The scanning ophthalmoscope further comprises a scan transfer device, wherein the scan transfer device is a reflective element and has two foci and the apparent point source is provided at a first focus of the scan transfer device and an eye is accommodated at a second focus of the scan transfer device, and wherein the scan transfer device transfers the two-dimensional collimated light scan from the apparent point source into the eye. The first and second scanning elements have operating parameters which are selected to control the direction of the two-dimensional collimated light scan from the apparent point source and/or adjust the dimensions of the two-dimensional collimated light scan from the apparent point source.

The present invention relates to a scanning laser ophthalmoscope (SLO) for scanning the retina of a human eye and a method of scanning the retina of a human eye. More particularly, the present invention relates to a scanning laser ophthalmoscope (SLO) for scanning the retina of a human eye and a method of scanning the retina of a human eye which involves the use of adaptive optics (AO) to compensate for wavefront aberrations caused by the eye and the SLO.

Cellular imaging in the living eye has been demonstrated to be possible using adaptive optic techniques (AO) originally derived from astronomy. Measuring and correction of the unwanted beam distortions introduced by the imperfect optics of the eye enables substantially higher resolution images of the retina to be obtained. Using AO techniques individual cones in the photoreceptor layer of the eye can be resolved. This greatly assists the ability to diagnose pathology in the eye.

Images of the resolution required are only possible by correcting the aberrations introduced by the human eye. To do this, it is necessary to measure these aberrations on the same plane where the pupil of the eye sits and correct them on the same plane. To achieve this, it is necessary to relay the image of the pupil to a different plane in space to perform the measurement and correction. The plane where an image of an object is formed is referred to as conjugate with the object. Hence, in this case it is necessary to create a conjugate plane of the eye pupil where the measurements can be performed, and a second one to perform the correction. A method of wavefront sensing, such as a Hartmann-Shack sensor, samples the wavefront, which is ideally planar in a perfect, collimated beam, across the pupil conjugate, which may be a telescopically replicated copy or image of the actual eye pupil, and reconstructs the aberrations at the pupil. The sensed wavefront aberrations are used to control an AO device, such as a deformable mirror, disposed in the optical path in order to compensate for the aberrations. This process of measurement and correction is done within a fast control loop so factors like dynamic tear film can be compensated out.

Additionally, photodetection needs to be carried out in a plane conjugate with the retina of the eye, and the detector's lens needs to sit on a plane conjugate with the pupil of the eye. Hence, in an AO system the location of the retinal and pupil conjugate planes is of central importance.

Scanning laser ophthalmoscopes (SLOs), such as those described in the Applicant's European Patent No. 0730428 and European Patent Application No. 07733214.6, are well established as an effective diagnostic tool for retinal imaging. Essentially, a laser beam of light is traversed across the retina with the returned energy being collected into a frame store to form an image. In contrast with the more conventional fundus camera, which floods light across the retina, the laser beam illuminates only a single pixel at a time, bringing benefits in terms of signal to noise and the ability to reject reflections from layers other than the diagnostic target. In an SLO system, laser light is relayed from one scan element to a second scan element and then into the eye. This coupling seeks to assure that a well formed beam enters the pupil with orthogonal, linear scans, low loss transmission and high efficiency conversion into the electronic domain.

Adaptive optics scanning laser ophthalmoscopes (AOSLOs), such as that described in U.S. Pat. No. 7,118,216 (to University of Rochester) are also known which are capable of obtaining high resolution images of the retina of the eye.

At a cellular scale with high magnification, which is a result of the 1 to 2 degree isoplanatic angle of the eye, it is necessary to take a large number of scans in order to build up an image of the key macular area of the retina. A montage of images is then created to obtain an overall image of the macular area of the retina.

While these known AOSLOs are capable of producing a montage of high resolution images of the key macular area of the retina, they are limited in that they are not capable of collecting the individual images at a fast enough rate to minimise movement artefact of the retina. For example, in order to achieve high resolution images in some known AOSLOs, it is necessary to reposition the patient's pupil relative to the AOSLO before every scan. This introduces a significant delay between each scan. Also, the repositioning of the patient's pupil introduces continuity errors between each scan. The result of this is that discontinuities, distortions and errors are introduced into the montage, which makes the ability to diagnose pathology in the eye more difficult. This also adds to the overall imaging session complexity and time.

Systems with non elliptical relays that intend to access a wider field of view must use off axis spherical relays in order to achieve a large field and manageable aberrations, the relay focal lengths must be very large, which results in overall system size that is not practical for transport and clinical environments.

It is an object of the present invention to provide a scanning laser ophthalmoscope (SLO) for scanning the retina of a human eye and a method of scanning the retina of a human eye which obviates or mitigates one or more of the disadvantages referred to above.

According to a first aspect of the present invention there is provided a scanning ophthalmoscope for scanning the retina of an eye comprising:
a source of collimated light;
a first scanning element;
a second scanning element;
   wherein the source of collimated light and the first and second scanning elements combine to provide a two-dimensional collimated light scan from an apparent point source; and
the scanning ophthalmoscope further comprises a scan transfer device, wherein the scan transfer device has two foci and the apparent point source is provided at a first focus of the scan transfer device and an eye is accommodated at a second focus of the scan transfer device, and wherein the scan transfer device transfers the two-dimensional collimated light scan from the apparent point source into the eye,
wherein the first and second scanning elements have operating parameters which are selected to control the direction of the two-dimensional collimated light scan from the apparent point source and/or adjust the dimensions of the two-dimensional collimated light scan from the apparent point source.

Selecting the operating parameters of the first and second scanning elements to control the direction of the two-dimensional collimated light scan and/or adjust the dimensions of the two-dimensional collimated light scan allows the size of the area and position of the scan on the retina to be controlled. For example, the first and second scanning elements may be configured to produce a “maximum area” two-dimensional collimated light scan. The operating parameters may then be selected to adjust the horizontal/vertical dimensions of the scan such that a “smaller area” scan may be produced at any point within the “maximum area” scan. This effectively allows the “smaller area” scan to be “moved” across the retina within the “maximum area” by an appropriate selection of the operating parameters to build up a montage of high resolution images of the retina.

Depending on the scanning elements used, the operating parameters can be selected to control the direction of the two-dimensional collimated light scan from the apparent point source and/or adjust the dimensions of the two-dimensional collimated light scan from the apparent point source. For example, if the scanning elements are rotating, or oscillating, elements, the direction of the two-dimensional collimated light scan from the apparent point source can be controlled. However, if scanning elements are line scanning elements (e.g. laser line scanner), the dimensions of the two-dimensional collimated light scan from the apparent point source can be controlled. It should be appreciated that a combination of rotating, oscillating and line scanning elements could be used as the first and second scanning elements in the SLO.

Importantly, the two-dimensional collimated light scan always emanates from the apparent point source, regardless of the selected operating parameters of the first and second scanning elements.

The first and second scanning elements may comprise an oscillating mechanism. The oscillating mechanism may be a resonant scanner.

The first and second scanning elements may comprise an oscillating plane mirror. The oscillating plane mirror may be a galvanometer mirror.

The first and second scanning elements may comprise a rotating mechanism. The rotating mechanism may comprise a rotating polygon mirror.

The first and second scanning elements may comprise a line scanning element. The line scanning element may comprise a laser line scanner. The laser line may be generated by a diffractive optical element, cylindrical lens, or other known means of creating a laser line.

The first and second scanning elements may comprise a combination of oscillating mechanisms, rotating mechanisms or line scanning elements, as described above.

The operating parameters of the first and second scanning elements may include the amplitude of the oscillation and the rotational offset of the oscillation. The operating parameters of the first and second scanning elements may also include the velocity of the oscillation.

The scanning ophthalmoscope may be able to produce up to 150 degree scans, for example 120 degrees, 110 degrees, 90 degrees, 60 degrees, 40 degrees, 20 degrees, of the retina of the eye, measured at the pupillary point of the eye. The scanning ophthalmoscope may be able to produce such scans of the retina of the eye, through a 2 mm undilated pupil of the eye. However, it should be appreciated that the SLO is also capable of producing scans of the retina of the eye through, for example, an 8 mm dilated pupil, as is known for AO measurements.

The oscillating mechanism may be able to produce variable angular amplitude of up to 10 degrees, for example 1 degree, 2 degrees, 3 degrees, 4 degrees, 5 degrees, 6 degrees, 7 degrees, 8 degrees, 9 degrees, 10 degrees.

The oscillating mechanism may also be able to produce variable angular amplitude of up to 40 degrees by adjusting the magnification and using a smaller oscillating mirror. This is effected by relay magnification.

The scan transfer device may comprise an elliptical mirror. The scan transfer device may comprise an aspherical mirror. The scan transfer device may comprise an ellipsoidal mirror. The scan transfer device may comprise a pair of parabola mirrors. The scan transfer device may comprise a pair of paraboloidal mirrors.

The source of collimated light may comprise a laser light source. The source of collimated light may comprise a light emitting diode, such as a fibre coupled super luminescent diode (SLD).

The source of collimated light are preferably intense, near infra-red, near spatially coherent and highly collimated.

The scanning ophthalmoscope may further comprise a scan relay device. The source of collimated light, the first and second scanning elements and the scan relay device combine to provide the two-dimensional collimated light scan from the apparent point source.

The scan relay device may comprise two foci. One foci of the scan relay device may be coincident with one foci of the scan transfer device.

The scan relay device may comprise an elliptical mirror. The scan relay device may comprise an aspherical mirror. The scan relay device may comprise an ellipsoidal mirror. The scan relay device may comprise a pair of parabola mirrors. The scan relay device may comprise a pair of paraboloidal mirrors.

The rotational axis of the second scanning element may be substantially parallel to a line joining the two foci of the scan transfer device. Alternatively, the rotational axis of the second scanning element may be substantially perpendicular to a line joining the two foci of the scan transfer device.

The rotational axis of the first scanning element may be substantially parallel to a line joining the two foci of the scan transfer device. Alternatively, the rotational axis of the first scanning element may be substantially perpendicular to a line joining the two foci of the scan transfer device.

In the provision of the two-dimensional collimated light scan from the apparent point source, the scan relay device may produce a one-dimensional collimated light scan, and the line joining the two foci of the scan transfer device may lie substantially on a plane defined by the one-dimensional collimated light scan produced by the scan relay device.

The rotational axis of the second scanning element may be within approximately 5 degrees of the line joining the two foci of the scan transfer device. The rotational axis of the second scanning element may be within approximately 2 degrees of the line joining the two foci of the scan transfer device. The rotational axis of the second scanning element and the line joining the two foci of the scan transfer device, may have a degree of parallelism which depends on chosen eccentricities of one or more components of the scanning ophthalmoscope. The rotational axis of the second scanning element and the line joining the two foci of the scan transfer device, may have a degree of parallelism determined by a user of the scanning ophthalmoscope, according to an acceptable level of shear in images of the retina produced by the ophthalmoscope.

The line joining the two foci of the scan transfer device may be within approximately 5 degrees of the plane defined by the one-dimensional collimated light scan produced by the scan relay device. The line joining the two foci of the scan transfer device may be within approximately 2 degrees of the plane defined by the one-dimensional collimated light scan produced by the scan relay device. The line joining the two foci of the scan transfer device and the plane defined by the one-dimensional collimated light scan produced by the scan relay device, may have a degree of coincidence which depends on chosen eccentricities of one or more components of the scanning ophthalmoscope. The line joining the two foci of the scan transfer device and the plane defined by the one-dimensional collimated light scan produced by the scan relay device, may have a degree of coincidence determined by a user of the scanning ophthalmoscope, according to an acceptable level of shear in images of the retina produced by the ophthalmoscope.

In the provision of the two-dimensional collimated light scan from the apparent point source, the scan relay device may produce a one-dimensional collimated light scan, and the line joining the two foci of the scan transfer device may be substantially perpendicular to a plane defined by the one-dimensional collimated light scan produced by the scan relay device.

The rotational axis of the first scanning element may be within approximately 5 degrees of the line joining the two foci of the scan transfer device. The rotational axis of the first scanning element may be within approximately 2 degrees of the line joining the two foci of the scan transfer device. The rotational axis of the first scanning element and the line joining the two foci of the scan transfer device, may have a degree of parallelism which depends on chosen eccentricities of one or more components of the scanning ophthalmoscope. The rotational axis of the first scanning element and the line joining the two foci of the scan transfer device, may have a degree of parallelism determined by a user of the scanning ophthalmoscope, according to an acceptable level of shear in images of the retina produced by the ophthalmoscope.

The components of the scanning ophthalmoscope are arranged such that the apparent point source is stationary at the pupil of the eye. This ensures that light reflected back from the retina of the eye is received back into the common optical path of the ophthalmoscope.

The scanning ophthalmoscope may further comprise:
a light detection device for detecting light reflected from the retina to produce an image of the scanned area of the retina.

The light detection device may comprise a photomultiplier or an avalanche photodiode (APD). The detectors are preferably low noise and high gain.

The scanning ophthalmoscope may further comprise:
a wavefront sensing device for detecting wavefront aberration in the reflected light in the common optical path; and
a wavefront compensation device including an adaptive optical element disposed in the common optical path between the source of collimated light and the eye for compensating the wavefront aberration in the reflected light.

The wavefront aberration in the reflected light may include aberrations introduced by the eye and/or the scanning ophthalmoscope. The wavefront aberrations introduced by the scanning ophthalmoscope may include aberrations introduced by the first scanning element, the second scanning element, the scan relay device or the scan transfer device.

The wavefront compensation device compensates for the aberrations introduced by the eye and/or the wavefront aberrations introduced by the first scanning element, the second scanning element, the scan relay device or the scan transfer device.

The wavefront sensing device may comprise a Hartmann-Shack detector or a Charge Coupled Device (CCD).

The adaptive optical element may comprise a deformable mirror.

According to a second aspect of the present invention there is provided a method of scanning the retina of an eye comprising the steps of:
providing a source of collimated light, a first scanning element and a second scanning element, wherein the first and second scanning elements have operating parameters;
using the source of collimated light and the first and second scanning elements in combination to provide a two-dimensional collimated light scan from an apparent point source;
selecting the operating parameters of the first and second scanning elements to control the direction of the two-dimensional collimated light scan from the apparent point source and/or to adjust the dimensions of the two-dimensional collimated light scan from the apparent point source;
providing a scan transfer device having two foci;
providing the apparent point source at a first focus of the scan transfer device and accommodating the eye at the second focus of the scan transfer device; and
using the scan transfer device to transfer the two-dimensional collimated light scan from the apparent point source to the eye.

Selecting the operating parameters of the first and second scanning elements to control the direction of the two-dimensional collimated light scan and/or adjust the dimensions of the two-dimensional collimated light scan allows the size of the area and position of the scan on the retina to be controlled. For example, the first and second scanning elements may be configured to produce a “maximum area” two-dimensional collimated light scan. The operating parameters may then be selected to adjust the horizontal/vertical dimensions of the scan such that a “smaller area” scan may be produced at any point within the “maximum area” scan. This effectively allows the “smaller area” scan to be “moved” across the retina within the “maximum area” by an appropriate selection of the operating parameters to build up a montage of high resolution images of the retina.

Depending on the scanning elements used, the operating parameters can be selected to control the direction of the two-dimensional collimated light scan from the apparent point source and/or adjust the dimensions of the two-dimensional collimated light scan from the apparent point source. For example, if the scanning elements are rotating, or oscillating, elements, the direction of the two-dimensional collimated light scan from the apparent point source can be controlled. However, if scanning elements are line scanning elements (e.g. laser line scanner), the dimensions of the two-dimensional collimated light scan from the apparent point source can be controlled. It should be appreciated that a combination of rotating, oscillating and line scanning elements could be used as the first and second scanning elements in the SLO.

The method of scanning the retina of the eye may also include providing a scan relay device, wherein the source of collimated light, the first and second scanning elements and the scan relay device combine to provided the two-dimensional collimated light scan from the apparent point source.

The scan relay device may comprise two foci and one of the foci of the scan relay device may be coincident with one foci of the scan transfer device.

The rotational axis of the second scanning element may be substantially parallel to a line joining the two foci of the scan transfer device. Alternatively, the rotational axis of the second scanning element may be substantially perpendicular to a line joining the two foci of the scan transfer device.

The rotational axis of the first scanning element may be substantially parallel to a line joining the two foci of the scan transfer device. Alternatively, the rotational axis of the first scanning element may be substantially perpendicular to a line joining the two foci of the scan transfer device.

In the provision of the two-dimensional collimated light scan from the apparent point source, the scan relay device may produce a one-dimensional collimated light scan, and the line joining the two foci of the scan transfer device may lie substantially on a plane defined by the one-dimensional collimated light scan produced by the scan relay device. Alternatively, in the provision of the two-dimensional collimated light scan from the apparent point source, the scan relay device may produce a one-dimensional collimated light scan, and the line joining the two foci of the scan transfer device may be substantially perpendicular to a plane defined by the one-dimensional collimated light scan produced by the scan relay device.

In the provision of the two-dimensional collimated light scan from the apparent point source, the scan compensation device produces a one-dimensional collimated light scan, and the line joining the two foci of the scan transfer device either lies substantially on a plane defined by the one-dimensional collimated light scan produced by the scan compensation device when the rotational axis of the second scanning element is parallel to the line joining the two foci of the scan transfer device, or is substantially perpendicular to the plane defined by the one-dimensional collimated light scan when the rotational axis of the second scanning element is perpendicular to the line joining the two foci of the scan transfer device.

The components of the scanning ophthalmoscope are arranged such that the apparent point source is stationary at the pupil of the eye. This ensures that light reflected back from the retina of the eye is received back into the common optical path of the ophthalmoscope.

The method of scanning the retina of an eye may include providing a light detection device for detecting light reflected from the retina and using the light detection device to produce an image of the scanned area of the retina.

The method of scanning the retina of an eye may include a providing wavefront sensing device for detecting wavefront aberration in the reflected light in the common optical path, and a wavefront compensation device including an adaptive optical element disposed in the common optical path between the source of collimated light and the eye, and using the wavefront compensation device to compensate for the wavefront aberration in the reflected light in the common optical path.

The wavefront aberration in the reflected light may include aberrations introduced by the eye and/or the scanning ophthalmoscope. The wavefront aberrations introduced by the scanning ophthalmoscope may include aberrations introduced by the first scanning element, the second scanning element, the scan relay device or the scan transfer device. The method of scanning the retina of an eye compensates for either or both of these aberrations to achieve high resolution images of the retina.

The method of scanning the retina of an eye may include the step of providing a program of predetermined selected operating parameters for the first and second scanning elements and following the program of predetermined selected operating parameters to produce a plurality of images of the retina.

The method of scanning the retina of an eye may include the step of combining at least a portion of the plurality of images of the retina to form a montage of the retina.

The method of scanning the retina of an eye allows a number of different areas of the retina to be scanned by effectively allowing the scan area to be moved across the retina. Therefore a number of high resolution images may be obtained and combined to provide a montage of high resolutions images of the retina.

The operating parameters of the first and second scanning elements may be driven under software control. This enables predictable, repeatable sub-scans to be obtained, with precise relationships in the assembled composite montage.

The method of scanning the retina of an eye may include the step of varying the amplitude of the angle of scan of the two-dimensional collimated light scan from the apparent point source. Varying the amplitude of the angle of scan of the two-dimensional collimated light scan from the apparent point source may be performed by adjusting the magnification between the scanning elements and the scan transfer device and scan relay device.

The combination of the first and second scanning elements, scan relay device, scan transfer device and adaptive optics described above enables a unique capability to capture narrow field of view retinal images at high resolution and to move that field of view across the retina to build up image montage sequences without adjustment of the subject's pupil position. This capability is assisted by, although not essential, the scan relay device and scan transfer device being ellipsoidal mirrors.

The montage of images is easier to obtain in this manner, as the pupil conjugate at the eye doesn't move, even with large scale scan change. The optical axis remains centred on the eye pupil and the image of that pupil relayed to the deformable mirror of the adaptive optical element does not move significantly either. Therefore, the measured aberrations for that portion of they eye lens do not move or change and thus the adaptive optical loop and the deformable mirror correction remains effective.

An embodiment of the present invention will now be described, by way of example only, with reference to the accompanying drawings, in which:

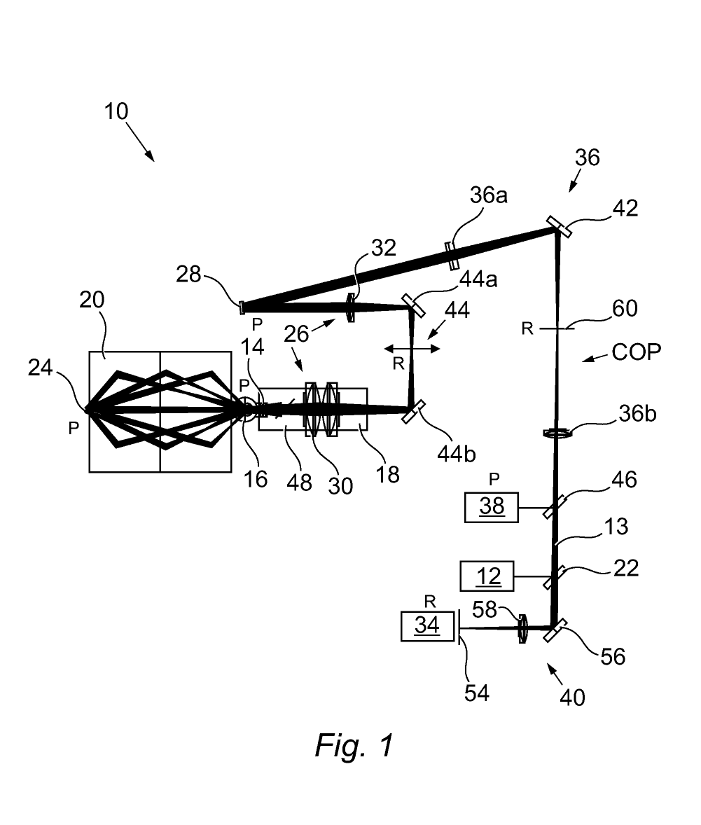

FIG. 1 is optical schematic a scanning laser ophthalmoscope (SLO) according to the present invention, which indicates the common optical path between a source of collimated light and a subject's eye;

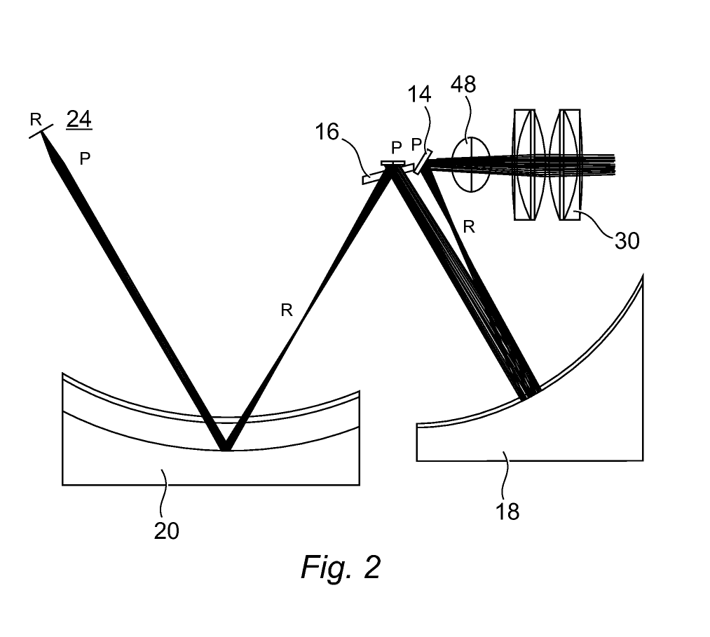

FIG. 2 is a 90 degree rotation of the SLO of FIG. 1, which indicates the surfaces of the scan transfer device and the scan relay device;

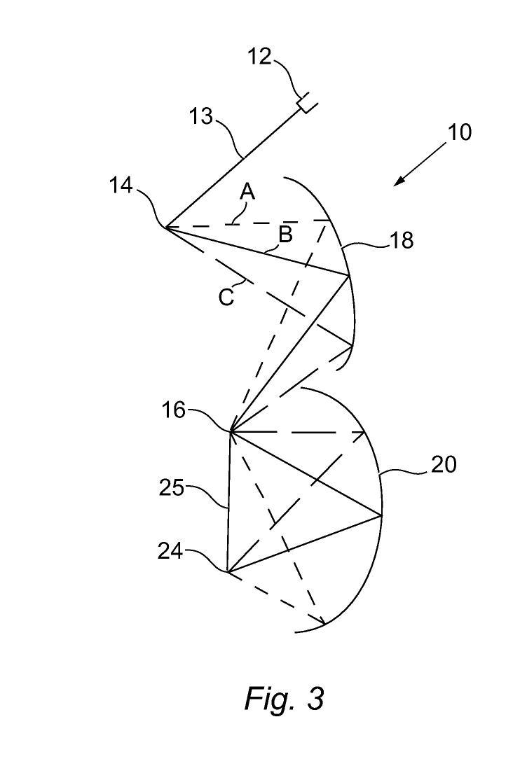

FIG. 3 is a simplified optical schematic of the source of collimated light, first and second scanning elements, scan relay device and scan transfer device of the SLO of FIG. 1, which indicates the scan path of collimated light between the first scanning element and the subject's eye;

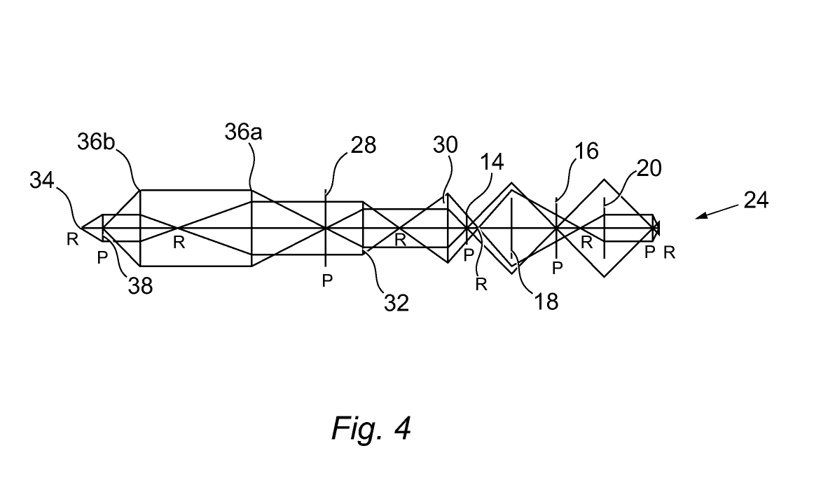

FIG. 4 is a schematic ray diagram of the SLO of FIG. 1;

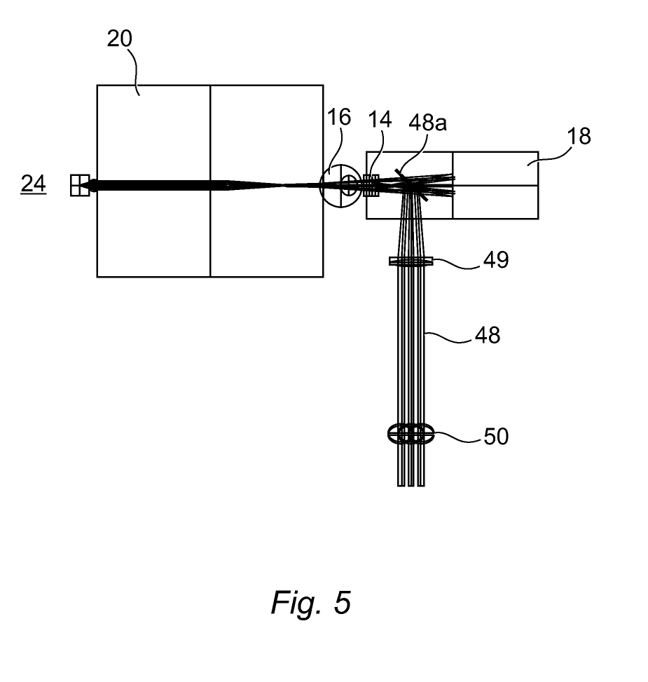

FIG. 5 is an optical schematic of the SLO of FIG. 1, which illustrates the wavefront sensing beacon;

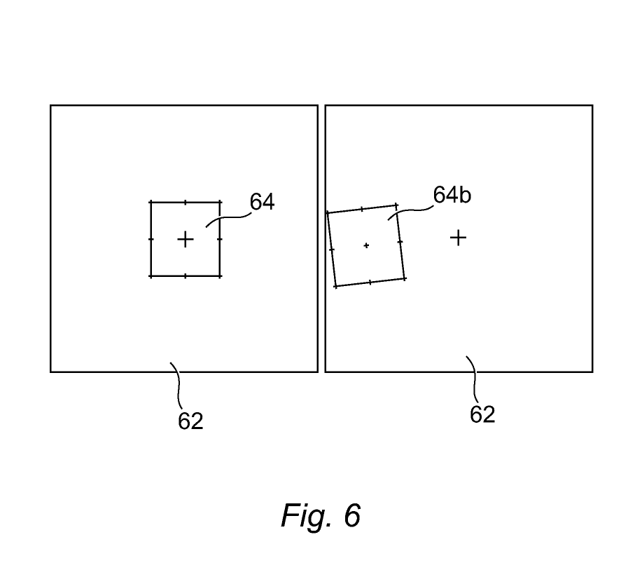

FIG. 6 is a schematic diagram illustrating the adjustment of the operating parameters of the first and second scanning elements; and

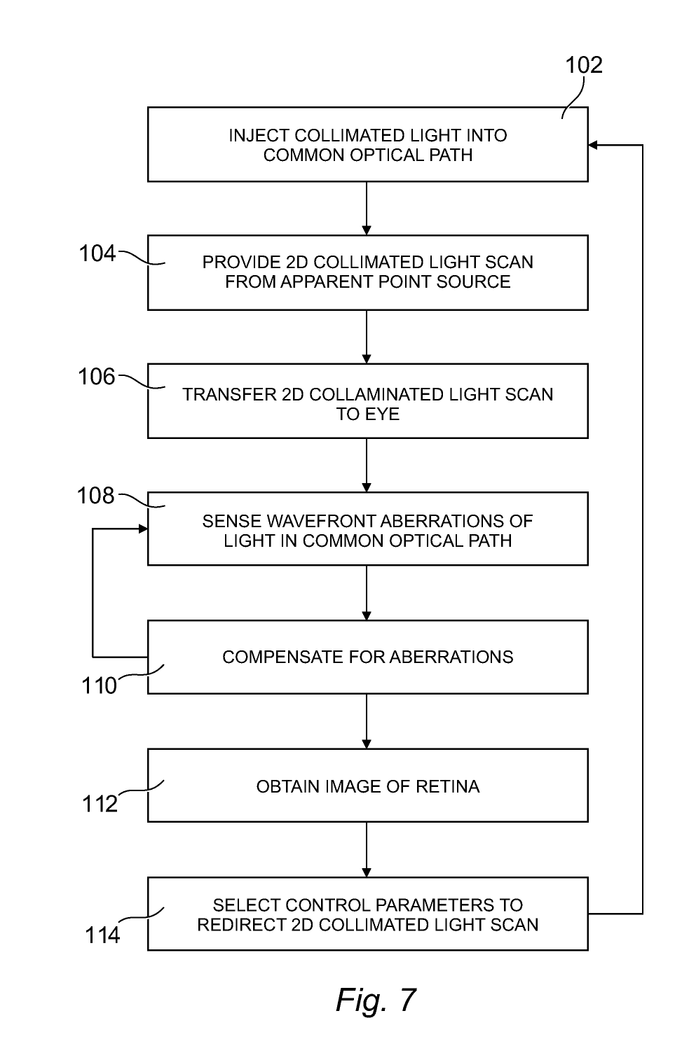

FIG. 7 is a flow chart of the operational steps carried out with the SLO of FIG. 1.

It should be noted that in FIGS. 1 to 4 points on the optical path which are conjugate to the pupil of the eye are labelled P, and points of the optical path which are conjugate to the retina of the eye are labelled R. With reference to FIGS. 1 to 3, the scanning laser ophthalmoscope (SLO) 10 including a source of collimated light 12, a first scanning element 14, a second scanning element 16, scan relay device 18 and scan transfer device 20.

In the embodiment described here the source of collimated light 12 is a super luminescent diode (SLD). However, it should be appreciated that any suitable source of collimated light could be used, such as a single frequency laser diode, vertical-cavity surface-emitting laser, or other source that has enough intensity and spatial coherence to be well collimated and produce adequate retinal illumination. An SLD was chosen to reduce speckle. The SLD may have at least 20 nm bandwidth. However, it should be appreciated that SLDs having bandwidths below or above 20 nm may also be used. The SLD is fibre coupled into polarisation maintaining fibre passed through a fibre coupled modulator (if required) to provide on/off modulation during sinusoidal fast scan. The laser beam 13 is injected into the system with a fibre collimator (not shown) with an output diameter of 6.5 mm. The fibre collimator is mounted on a tip/tilt mount with rotation. The rotation of the collimator is needed to set in the input polarisation to achieve 90/10 (reflecting 10% of linear polarised light into the system and transmitting 90% of the reflected return) beam splitting at injection. The laser is aligned into the system using the tip/tilt on its mount and tip/tilt on the mount of a beam splitter 22. Fibre coupled devices facilitate easy alignment and replacement.

The source of collimated light 12 is preferably intense, near infra-red and spatially coherent to produce a highly collimated beam.

The beam splitter 22 is an uncoated BK7 window, 5 mm thick and oriented at 45 degrees to the laser beam 13 from the SLD. The back side of the beam splitter 22 is anti-reflection to reduce back reflections into the detectors (see below).

High efficiency coatings with wavelength optimisation for minimisation of back reflections, spatial filtering and adequate aperture control all form an important part of the design of the system.

The first scanning element 14 is a slow speed oscillating plane mirror, such as a galvanometer mirror, and the second scanning element 16 is a resonant scanner, such as a resonant scanning mirror. The galvanometer mirror 14 and the resonant scanning mirror 16 axes are arranged orthogonally to create a two-dimensional collimated light scan, in the form of a raster scan pattern of the laser beam 13.

The galvanometer mirror 14 provides a one-dimensional collimated light scan, which, in the embodiment described here, comprises a vertical one-dimensional scan of the laser beam 13. This generates a vertical scan component of the raster scan pattern.

The resonant scanner 16 provides a plurality of second one-dimensional collimated light scans, which, in this embodiment of the invention, comprises horizontal one-dimensional scans of the laser beam 13. Each oscillation of the resonant scanner 16 generates a horizontal scan component of the raster scan pattern.

The rotational axis of the galvanometer mirror 14 is perpendicular to the resonant scanner 16.

FIG. 3 illustrates the path of the laser beam 13 in a horizontal one-dimensional scan produced by one oscillation of the galvanometer mirror 14. Path A is an example of the laser beam reflected from the galvanometer mirror 14 at the start of the rotation; path B is an example of the laser beam reflected from the galvanometer mirror 14 at an intermediate point of the rotation; and path C is an example of the laser beam reflected from the galvanometer mirror 14 at the end of the rotation.

The galvanometer mirror 14 and the resonant scanner 16 thus together create a two-dimensional collimated light scan in the form of a raster scan pattern.

The galvanometer mirror 14 and the resonant scanner 16 have operating parameters which include the amplitude of the oscillation and the rotational offset of the oscillation. The operating parameters also include the velocity of oscillation. Both of these operating parameters may be selected to control the direction of the two-dimensional collimated light scan from the apparent point source.

The resonant scanner 16 is capable of producing variable angular amplitude of up to 10 degrees, for example 1 degree, 2 degrees, 3 degrees, 4 degrees, 5 degrees, 6 degrees, 7 degrees, 8 degrees, 9 degrees, 10 degrees. The resonant scanner 16 is capable of producing these various amplitudes of oscillation at any point relative to its rotational axis. That is, the resonant scanner 16 can produce up to 10 degrees of variable angular amplitude at any point within its 360 degrees of revolution.

The resonant scanner 16 is housed in a rotation mount (not shown) that can adjust the centring (or eccentricity) of the scanned laser beam 13 on the retina, which provides the ability to “move” the imaging field across the retina.

The galvanometer mirror 14 is also capable of producing variable angular amplitude of up to 80 degrees, for example 10 degree, 20 degrees, 30 degrees, 40 degrees, 5 degrees, 60 degrees, 70. The galvanometer mirror 14 is capable of producing these various amplitudes of oscillation at any point relative to its rotational axis. That is, the galvanometer mirror 14 can produce up to 80 degrees of variable angular amplitude at any point within its 360 degrees of revolution. Note that the angular amplitude of up to 80 degrees is an “optical” angle. This translates to 40 degrees “mechanical” angle.

The scan relay device 18 has two foci. In the embodiment described here the scan relay device 18 is an ellipsoidal mirror, and is referred to as a slit mirror. It should be appreciated, however, that the scan relay device 18 may have an alternative form.

The galvanometer mirror 14 is positioned at a first focus of the slit mirror 18 and the resonant scanner 16 is positioned at the second focus of the slit mirror 18.

The scan transfer device 20 is an aspherical mirror in the form of an ellipsoidal mirror, and is referred to as a main mirror. The main mirror 20 has two foci. In the embodiment described and illustrated here, the main mirror 20 is configured to provide a 40 degree field of view in both the vertical and horizontal directions (i.e. 40 degree×40 degree) on the retina. However, it should be appreciated that the main mirror 20 may be configured to provide a 200 degree field of view (external angle) in both the vertical and horizontal directions (i.e. 200 degree×200 degree) on the retina.

The resonant scanner 16 is also positioned at a first focus of the main mirror 20. A subject's eye 24 is positioned at a second focus of the main mirror 20.

The laser beam 13 is thus conveyed to the subject's eye 24, via the galvanometer mirror 14, the slit mirror 18, the resonant scanner 16 and the main mirror 20. The galvanometer mirror 14, the slit mirror 18, and the resonant scanner 16, combine to provide a two-dimensional collimated light scan, in the form of a raster scan pattern, from an apparent point source. This is coupled from the resonant scanner 16 to the subject's eye 24, by the main mirror 20.

The scanning ophthalmoscope may be able to produce up to 150 degree scans, for example 120 degrees, 110 degrees, 90 degrees, 60 degrees, 40 degrees, 20 degrees, of the retina of the eye, measured at the pupillary point of the eye. The scanning ophthalmoscope may be able to produce such scans of the retina of the eye, through a 2 mm undilated pupil of the eye. However, it should be appreciated that the SLO is also capable of producing scans of the retina of the eye through, for example, an 8 mm dilated pupil, as is known for AO measurements.

The components of the SLO 10 are arranged such that the apparent point source is stationary at the pupil of the eye. This ensures that a beam of reflected light from the retina of the subject's eye 24 is conveyed back through the optical path of the SLO 10. The reflected light is used to produce an image of the subject's retina in the known manner.

The scan relay device 18 transfers the scan of the laser beam 13 from the galvanometer mirror 14 to the resonant scanner 16. The scan relay device 18 provides point to point transfer, without introducing any translational component, which would cause failure of the laser beam 13 to enter through the pupil of the subject's eye 24. Thus the laser beam 13 appears to come from an apparent point source.

Since the galvanometer mirror 14 is positioned at the first focus of the slit mirror 18, light from the galvanometer mirror 14 will always be reflected through the second focus of the slit mirror 18, regardless of the angle of deflection of light from the galvanometer mirror 14 onto the slit mirror 18. The effect of this is that the raster scan pattern of the laser beam 13 is transmitted without disruption through the pupil of the subject's eye 24.

This enables ultra-wide retinal images of the retina to be obtained, as is known in the art.

Judicious matching of eccentricities of the slit mirror 18 and the main mirror 20 provides well behaved deviation from perfect scan linearity. Symmetric deviation, as a function of angle from the optic axis of the eye, enables simple compensation of distance measurements on the retina in software, and an adequately intuitive retinal display representation.

In the embodiment of the invention described and illustrated here the components of the SLO 10 are arranged such that the rotational axis of the resonant scanner 16 is substantially parallel to a line 25 joining the two foci of the main mirror 20, such that the laser beam 13 is scanned across the secondary axis of the slit mirror 18. Furthermore, in the provision of the two-dimensional collimated light scan from the apparent point source, the galvanometer mirror 14 produces a one-dimensional scan which is incident on the slit mirror 18. The slit mirror 18 also therefore produces a one-dimensional scan. The components of the SLO 10 are arranged such that the line 25 joining the two foci of the main mirror 20 lies substantially on a plane defined by the one-dimensional scan produced by the slit mirror 18. This arrangement of components offers a number of advantages.

The key advantage of having the rotational axis of the resonant scanner 16 and the galvanometer mirror 14 being substantially parallel to the line 25 joining the two foci of the main mirror 20, and the line 25 lying substantially on the plane defined by the one-dimensional scan produced by the slit mirror 18, is that the scanned image of the subject's retina does not have, or has a reduced, “shear” component. This is because the arrangement of the components of the SLO 10 removes the requirement to provide a “tilt” to the input laser beam 13, thus improving orthogonality between the horizontal and vertical components of the two-dimensional scan and the line 25 joining the two foci of the main mirror 20.

Therefore, it is possible to measure consistent dimensions within retinal images, thus facilitating simpler quantification of feature size within these images.

Preferably, there is no offset rotation about the axis perpendicular to the rotational axis of the resonant scanner 16 and the galvanometer mirror 14, as this would introduce distortions to the scan.

A further advantage of the arrangement of the components of the SLO 10 of the present invention, is that since all the components of the SLO 10 can lie in a single plane, manufacturing of the SLO 10 is simplified, which reduces build time and cost. Furthermore, this arrangement allows greater flexibility in the positioning of the subject's head in relation to the SLO 10.

Another advantage is that the number of components comprised in the SLO 10 of the present invention may be reduced, in comparison to previous ophthalmoscopes. This increases the optical brightness of the ophthalmoscope of the present invention, which is important when obtaining retinal images.

With reference to FIGS. 1, 2 and 4, a lens telescope relay 26 images the galvanometer mirror 14 to a deformable mirror 28 (an example of adaptive optical element). The telescope relay 26 comprises a first lens 30 (which is a doublet achromatic lens, see FIGS. 1 and 2) positioned at its front focal length from the galvanometer mirror 14, as illustrated in FIG. 4, and a second lens 32 spaced to collimate the output of the telescope relay 26, as illustrated in FIGS. 1 and 4. The deformable mirror 28 is placed at the back focal length of the second lens 32, as illustrated in FIG. 4. The telescope relay 26 additionally converts the retinal conjugate (R) so that it can be subsequently relayed to an imaging detector 34 (an example of a light detection device), as illustrated in FIGS. 1 and 4. The lens telescope relay 26 also relays the image to the galvanometer mirror 14 and produces the correct focal state into the slit mirror 18 and main mirror 20 system.

The magnification of the lens telescope relay 26 in the embodiment described here is 1:2, yielding a 6.5 mm wavefront sensor aperture matched to a 13 mm deformable mirror aperture. This permits only one telescope to relay the input and output laser beams. Using the same telescope for input and output eliminates the need for a separate relay. This provides a more light efficient system because of less optical surfaces than in standard AO systems.

The SLO 10 system design permits some degree of choice on the size of the deformable mirror 28. Components between the main mirror 20 and the first lens 30 remain identical for different sized deformable mirrors. The input/output relay telescope and the first lens 32 must change for different deformable mirror sizes. This would also result in a different laser and detector location and alignment.

A second lens telescope relay 36 comprising first and second achromatic lenses 36a, 36b) relays the deformable mirror 28, wavefront sensor 38 (see below) and retinal imaging optics 40.

A fold mirror 42 is placed between the two achromatic lenses 36a and 36b to reduce the size of the optical layout. An aperture 60 is also located between the fold mirror 42 and the second achromatic lens 36b. The fold mirror 42 reduces back reflections on the wavefront sensor 38 from the cornea of the eye.

In the embodiment described and illustrated here the SLO 10 includes a Badal focus system 44 (Badal optometer) to relieve the deformable mirror 28 of large focus correction requirement. The Badal focus system 44 incorporates a moveable stage to vary the path length between the lenses in the lens telescope relay 26. The Badal focus system 44 includes two mirrors 44a and 44b. The Badal focus system 44 here has increased focus range to meet 8D-12D correction. The output of the Badal focus system 44 is convergent to create the correct beam vergence for the slit mirror 18 and main mirror 20. To achieve lower spherical aberration two identical doublets are used.

As illustrated in FIG. 1, the wavefront sensor 38 (an example of a wavefront sensing device) is positioned adjacent the source of collimated light 12 and works with beam splitter 46 to detect wavefront aberration in the reflected light in the common optical path COP. The wavefront sensor 38 measures the aberrations on the same plane as the pupil of the eye, as illustrated in FIGS. 1 and 4. As described above, a conjugate of the plane of the pupil of the eye is created to perform the measurement of the aberration.

The wavefront sensor 38, which may be a Hartmann-Shack sensor, samples the wavefront across the pupil conjugate and reconstructs the aberrations at the pupil. Alternatively, the wavefront sensor 38 could be a Charge Coupled Device (CCD).

The input path for the source of collimated light and the wavefront sensing are separated. That is, there are two separate lasers used in the system. One laser (laser beam 13) is used to image the retina (Imaging Laser), and another laser (beacon laser 48) is used to sense the aberrations (Sensing Laser).

In the embodiment described here the input for the wavefront sensing is performed by a wavefront sensing beacon laser 48 located between the lens telescope relay 26 and the galvanometer mirror 14, as illustrated in FIGS. 1, 2 and 5.

To eliminate back reflections from the lenses 30, 32 in the lens telescope relay 26 and the Badal focus system 44, the beacon laser 48 is injected after the Badal focus system 44.

The beacon laser 48 is a 910 nm laser which is fibre coupled into polarisation maintaining fibre. It is collimated via lens 49 to 3.2 mm (1 mm at the eye) and is mounted on a translation stage 50 to move the beam off axis at the pupil of the eye to eliminate back reflections from the pupil of the eye. The mount also provides rotation to set the polarisation axis. The focal sate of the beacon laser 48 is set with a lens positioned to correspond with the system focal point that lies after the galvanometer mirror 14.

The deformable mirror 28 is also located at a conjugate of the plane of the pupil of the eye, as illustrated in FIGS. 1 and 4. The wavefront sensor 38 uses the measured aberrations to control the deformable mirror 28 correct for the aberrations. The process of measuring and correction is iterated in a fast control loop to an acceptable level.

The wavefront compensation device compensates for the aberrations introduced by the eye and/or the wavefront aberrations introduced by the first scanning element 14, the second scanning element 16, the scan relay device 18 or the scan transfer device 20.

The main axis of the main mirror 20 is arranged with the galvanometer mirror 14 so that dynamic correction can be applied, modifying the deformable mirror 28 synchronously with the scan position. A synchronization signal is relayed from the slow scan driver to the deformable mirror controller to update the mirror shape during the slow scan.

The compensated light from the retina is focused through a confocal aperture 54 and detected with the image detector 34. The image detector 34 is an avalanche photodiode (ADP). However, the image detector 34 may alternatively be a photomultiplier or other hybrid device capable of detecting low light levels at high speed, or the like. Before the compensated light reaches the confocal aperture 54 it is passed through a fold mirror 56 and an achromatic lens 58, as illustrated in FIGS. 1 and 4.

The image detector 34 therefore obtains high resolution images of the retina of the eye. By adjusting the focal plane of the imaging device, the confocal aperture acts to block light from out of focus layers, providing a depth sectioning capability as is done in standard SLO and microscope applications.

As described above, the galvanometer mirror 14 and the resonant scanner 16 have operating parameters which include the amplitude of oscillation and the rotational offset of the oscillation. These parameters may be selectively operable to control the direction of the two-dimensional collimated light scan from the apparent point source.

FIG. 6 illustrates an example of how the direction of the two-dimensional scan from the apparent point source can be adjusted to move the scan area across the retina. An area 62 is illustrated, which in the embodiment described and illustrated here, represents an approximate 40 degree field of view in both the vertical and horizontal directions (i.e. 40 degree×40 degree) on the retina. The area of the two-dimensional scan 64 on the left image represents an approximate 8 degree field of scan in both the vertical and horizontal directions (i.e. 8 degree×8 degree) on the retina. The scan 64 is the left image is “on axis”. If the control parameters of the galvanometer mirror 14 and the resonant scanner 16 are adjusted (i.e. the amplitude of oscillation and/or the rotational offset of the oscillation are varied), the scan 64 moves within the area 62, as illustrated in the right image, where the two-dimensional scan 64b has moved such that it is “off axis”. The scan 64b is rotated slightly due to the oblique angle of incidence on the scanning elements. This slight rotation can be corrected for with digital processing.

Varying the operating parameters of the scanning elements 14, 16 therefore allows the direction of the scan to be controlled such that the scan can move anywhere on the larger area 62. This allows the narrow scan area 64 to move across the retina to build up high resolution image montage sequences. Importantly, as the narrow scan area 64 moves across the larger area 62 (i.e. retina), the two-dimensional scan always comes from the apparent point source, regardless of its position relative to the larger area 62 (i.e. retina) and selected operating parameters.

The operating parameters of the first and second scanning elements may be driven under software control. This enables, predictable, repeatable narrow field scans to be obtained, with precise relationships in the assembled montage.

The SLO 10 operates as illustrated in FIG. 7. In step 102 the source of collimated light injects the laser beam 13 into the common optical path COP. In step 104 the scanning elements and scan relay device 18 combine to provide a two-dimensional collimated light scan from an apparent point source. In step 106 the scan transfer device 20 transfers the two-dimensional collimated light scan from its first focus to an eye accommodated at its second focus. In step 108 the wavefront 38 senses the wavefront aberrations of the light in the common optical path. In step 110 the wavefront sensor 38 compensates for the aberrations sensed in step 108 using the deformable mirror 28. Steps 108 and 110 can be performed in a loop (i.e. iteratively). In step 112 the image detector 34 obtains a high resolution image of the retina of the eye. In step 114 the control parameters are selected to direct the two-dimensional collimated light scan to a new area on the retina and steps 102 to 112 are repeated. Steps 102 to 114 are repeated as often as necessary to build up enough images to create a montage of the retina. The operation of the SLO 10 may include an additional step (not shown) of adjusting the scan amplitude angle of the two-dimensional collimated light scan by the methods described above.

The SLO 10 and method of scanning the retina of an eye of the present invention therefore obviate or mitigate the disadvantages of previous proposals by enabling a plurality of high resolution narrow field of view retinal images to be obtained across a significant portion of the retina without adjustment of the subject's pupil position. Obtaining such high resolution narrow field of view retinal images in this manner reduces the time delay between each scan and avoids the need to reposition the subject's pupil. The result of this is that the montage of images has less discontinuities, distortions and errors, which improves the quality of the image and increases the ability to diagnose pathology in the eye. The present invention also reduces the overall imaging session complexity and time, which results in fast automatic montage capture. The ellipsoidal relay allows for a compact system with wide field and manageable relays.

Modifications and improvements may be made to the above without departing from the scope of the present invention. For example, although the resonant scanner 16 has been described above as being capable of producing variable angular amplitude of up to 10 degrees, for example 1 degree, 2 degrees, 3 degrees, 4 degrees, 5 degrees, 6 degrees, 7 degrees, 8 degrees, 9 degrees, 10 degrees, it should be appreciated that a resonant scanner could be used which is capable of producing variable angular amplitude of up to 360 degrees. That is, the resonant scanner 16 is capable of producing amplitudes of oscillation of up to 360 degrees at any point relative to its rotational axis. That is, the resonant scanner 16 can produce up to 360 degrees of variable angular amplitude at any point within its 360 degrees of revolution.

Alternatively the magnifications of the elliptical relays may be adjusted to adjust the angular magnification to compensate for reduced mechanical scan angle of either scanner.

Also, although ellipsoidal coupling mirrors 18, 20 have been described and illustrated above, it should be appreciated that other coupling element may be used, such as diffractive elements, free form mirror surfaces or conventional lens relays, given the discrete wavelengths of the imaging system. Mirrors are better because of the reduction of chromatic effects from refractive coatings.

Furthermore, although a deformable mirror 28 has been illustrated and described above as being the adaptive optical element, it should be appreciated that other suitable adaptive optical elements could be used, such as deformable liquid lens devices, liquid crystal spatial light modulators, or other devices capable of altering the phase of incident light.

Also, the SLO 10 has been described and illustrated above as including scan relay device (slit mirror 18), it should be appreciated that this element is not essential and it is possible for the SLO 10 to provide the same advantages as described above without this component. Removing this component requires the laser beam to be “tilted” within the SLO, which causes some shearing effects on the images obtained. However, such an SLO is still capable of providing the two-dimensional scan from the apparent point source, regardless of its position relative to the larger area 62 (i.e. retina) and selected operating parameters.

Furthermore, although the first and second scanning elements 14 and 16 have been described and illustrated above as being a galvanometer mirror and a resonant scanner, respectively, it should be appreciated that other suitable scanning elements could be used, such as line scanning produced with a laser line source, or equivalent. Line scanning could be used as an effective alternative to point scanning. Here a line source produces a line illumination on the retina which is scanned orthogonally by a slow scanner. The line illumination is detected by a linear pixel array and a 2D image is built up by rotating the slow scanner.

Also, although the slit mirror 18 has been described above as being an ellipsoidal mirror having two foci, it should be appreciated that the scan relay device could take other forms. For example, the scan relay device could comprise an elliptical mirror, a pair of parabolic mirrors, a pair of paraboloidal mirrors or a combination of any of these components. The common technical feature provided by any of these component arrangements is that the scan relay device comprises two foci and produces a one-dimensional collimated light scan.

Where elliptical components are used in the scan relay device, it may also be necessary to provide beam compensation elements, such as cylindrical lenses.

Further, although the above described arrangement of the SLO 10 has the galvanometer mirror 14 positioned at the first focus of the slit mirror 18 and the resonant scanner 16 located at the second focus of the slit mirror 18, it should be appreciated that the position of the galvanometer mirror 14 and the resonant scanner 16 may be switched without affecting the operation of the SLO 10.

Furthermore, although the galvanometer mirror 14 has been described above as providing vertical scanning of the laser beam 13 and the resonant scanner 16 providing horizontal scanning, it should be appreciated that the axes of rotation and oscillation of these two elements could be switched, such that the galvanometer mirror 14 provides the horizontal scanning of the laser beam 13 and the resonant scanner 16 provides the vertical scanning. Therefore, the rotational axis of the second scanning element may be substantially parallel to the line joining the two foci of the scan transfer device and the line joining the two foci of the scan transfer device may lie substantially on the plane defined by the one-dimensional collimated light scan produced by the scan relay device; or the rotational axis of the second scanning element may be substantially perpendicular to the line joining the two foci of the scan transfer device and the line joining the two foci of the scan transfer device may be substantially perpendicular to the plane defined by the one-dimensional collimated light scan produced by the scan relay device.

In addition, although the above embodiment of the present invention has been described as providing 120 degree optical scans, it should be appreciated that the ophthalmoscope 10 may be configured to provide a lesser or greater angle of optical scan. As described above, this may be achieved, for example, by varying selection of the portion of the slit mirror 18 that the laser beam 13 is scanned across.

Also, the scan transfer device may comprise an elliptical mirror. The scan transfer device may comprise a pair of parabola mirrors. The scan transfer device may comprise a pair of paraboloidal mirrors.

Also, the rotational axis of the second scanning element may be within approximately 5 degrees of the line joining the two foci of the scan transfer device. The rotational axis of the second scanning element may be within approximately 2 degrees of the line joining the two foci of the scan transfer device. The rotational axis of the second scanning element and the line joining the two foci of the scan transfer device, may have a degree of parallelism which depends on chosen eccentricities of one or more components of the scanning ophthalmoscope. The rotational axis of the second scanning element and the line joining the two foci of the scan transfer device, may have a degree of parallelism determined by a user of the scanning ophthalmoscope, according to an acceptable level of shear in images of the retina produced by the ophthalmoscope.

Also, the rotational axis of the first scanning element may be within approximately 5 degrees of the line joining the two foci of the scan transfer device. The rotational axis of the first scanning element may be within approximately 2 degrees of the line joining the two foci of the scan transfer device. The rotational axis of the first scanning element and the line joining the two foci of the scan transfer device, may have a degree of parallelism which depends on chosen eccentricities of one or more components of the scanning ophthalmoscope. The rotational axis of the first scanning element and the line joining the two foci of the scan transfer device, may have a degree of parallelism determined by a user of the scanning ophthalmoscope, according to an acceptable level of shear in images of the retina produced by the ophthalmoscope.

Furthermore, the line joining the two foci of the scan transfer device may be within approximately 5 degrees of the plane defined by the one-dimensional collimated light scan produced by the scan relay device. The line joining the two foci of the scan transfer device may be within approximately 2 degrees of the plane defined by the one-dimensional collimated light scan produced by the scan relay device. The line joining the two foci of the scan transfer device and the plane defined by the one-dimensional collimated light scan produced by the scan relay device, may have a degree of coincidence which depends on chosen eccentricities of one or more components of the scanning ophthalmoscope. The line joining the two foci of the scan transfer device and the plane defined by the one-dimensional collimated light scan produced by the scan relay device, may have a degree of coincidence determined by a user of the scanning ophthalmoscope, according to an acceptable level of shear in images of the retina produced by the ophthalmoscope.

Also, although not illustrated above, in an optional step of FIG. 7 the retina can be scanned in an axial manner to produce a three-dimensional image.

Furthermore, although the first and second scanning elements have been described and illustrated above as oscillating mirrors, it should be appreciated that the first and second scanning elements may comprise line scanning elements. The line scanning element may comprise a laser line scanner. The laser line may be generated by a diffractive optical element, cylindrical lens, or other known means of creating a laser line.

Also, although the scanning elements have been described above as having operating parameters which allow the direction of the two-dimensional collimated light scan from the apparent point source can be controlled, it should be appreciated that if the scanning elements are line scanning elements (e.g. laser line scanner), the operating parameters are operable to adjust the dimensions (i.e. horizontal/vertical) of the two-dimensional collimated light scan from the apparent point source. This allows the size and position of the scan area to be adjusted, and hence effectively “moved” around the retina to obtain a montage of images thereof. Where line scanning elements are used, it is important to note that the detection and AO layout architecture is also modified, as is known in the art.

What we claimed is:

1. A scanning ophthalmoscope for scanning the retina of an eye comprising:
a source of collimated light;
a first scanning element;
a second scanning element;
wherein the source of collimated light and the first and second scanning elements combine to provide a two-dimensional collimated light scan from an apparent point source;
and the scanning ophthalmoscope further comprises a scan transfer device, wherein the scan transfer device is a reflective element and has two foci and the apparent point source is provided at a first focus of the scan transfer device and an eye is accommodated at a second focus of the scan transfer device, and wherein the scan transfer device transfers the two-dimensional collimated light scan from the apparent point source into the eye,
wherein the first and second scanning elements have operating parameters which are selected to control the direction of the two-dimensional collimated light scan from the apparent point source and/or adjust the dimensions of the two-dimensional collimated light scan from the apparent point source.

2. A scanning ophthalmoscope as claimed in claim 1, wherein the first and second scanning elements each comprise an oscillating mechanism and the operating parameters of the first and second scanning elements includes the amplitude of the oscillation, the velocity of the oscillation or the rotational offset of the oscillation.

3. A scanning ophthalmoscope as claimed in claim 1, wherein the scan transfer device comprises an asphehcal mirror, an ellipsoidal mirror, a pair of parabola mirrors or a pair of paraboloidal mirrors.

4. A scanning ophthalmoscope as claimed in claim 1, wherein the scanning ophthalmoscope further comprises a scan relay device and wherein the source of collimated light, the first and second scanning elements and the scan relay device combine to provide the two-dimensional collimated light scan from the apparent point source.

5. A scanning ophthalmoscope as claimed in claim 4, wherein the scan relay device comprises two foci and one foci of the scan relay device is coincident with one foci of the scan transfer device.

6. A scanning ophthalmoscope as claimed in claim 4, wherein the scan relay device comprises an elliptical mirror, an aspherical mirror, and ellipsoidal mirror, a pair of parabola mirrors or a pair of paraboloidal mirrors.

7. A scanning ophthalmoscope as claimed in claim 1, wherein the rotational axis of the second scanning element is substantially parallel or perpendicular to a line joining the two foci of the scan transfer device.

8. A scanning ophthalmoscope as claimed in claim 1, wherein the rotational axis of the first scanning element is substantially parallel or perpendicular to a line joining the two foci of the scan transfer device.

9. A scanning ophthalmoscope as claimed in claim 4, wherein, in the provision of the two-dimensional collimated light scan from the apparent point source, the scan relay device produces a one-dimensional collimated light scan, and the line joining the two foci of the scan transfer device either lies substantially on a plane defined by the one-dimensional collimated light scan produced by the scan relay device or perpendicular to the plane defined by the one-dimensional collimated light scan produced by the scan relay device.

10. A scanning ophthalmoscope as claimed in claim 1, wherein the scanning ophthalmoscope further comprises a light detection device for detecting light reflected from the retina to produce an image of the scanned area of the retina.

11. A scanning ophthalmoscope as claimed in claim 1, wherein the scanning ophthalmoscope further comprises a wavefront sensing device for detecting wavefront aberration in the reflected light in the common optical path and a wavefront compensation device including an adaptive optical element disposed in the common optical path between the source of collimated light and the eye for compensating the wavefront aberration in the reflected light.

12. A scanning ophthalmoscope as claimed in claim 11, wherein the wavefront sensing device comprises a Hartmann-Shack detector.

13. A scanning ophthalmoscope as claimed in claim 11, wherein the adaptive optical element comprises a deformable mirror.

14. A method of scanning the retina of an eye comprising the steps of:
providing a source of collimated light, a first scanning element and a second scanning element, wherein the first and second scanning elements have operating parameters;
using the source of collimated light and the first and second scanning elements in combination to provide a two-dimensional collimated light scan from an apparent point source;
selecting the operating parameters of the first and second scanning elements to control the direction of the two-dimensional collimated light scan from the apparent point source and/or to adjust the dimensions of the two-dimensional collimated light scan from the apparent point source;
providing a scan transfer device having two foci, wherein the scan transfer device is a reflective element; providing the apparent point source at a first focus of the scan transfer device and accommodating the eye at the second focus of the scan transfer device; and
using the scan transfer device to transfer the two-dimensional collimated light scan from the apparent point source to the eye.

15. A method of scanning the retina of an eye as claimed in claim 14, wherein the method includes the further step of providing a scan relay device and wherein the source of collimated light, the first and second scanning elements and the scan relay device combine to provided the two-dimensional collimated light scan from the apparent point source.

16. A method of scanning the retina of an eye as claimed in claim 15, wherein the scan relay device comprises two foci and one of the foci of the scan relay device is coincident with one foci of the scan transfer device.

17. A method of scanning the retina of an eye as claimed in claim 14, wherein the rotational axis of the second scanning element is substantially parallel or perpendicular to a line joining the two foci of the scan transfer device.

18. A method of scanning the retina of an eye as claimed in claim 14, wherein the rotational axis of the first scanning element is substantially parallel or perpendicular to a line joining the two foci of the scan transfer device.

19. A method of scanning the retina of an eye as claimed in claim 15, wherein in the provision of the two-dimensional collimated light scan from the apparent point source, the scan relay device produces a one-dimensional collimated light scan, and the line joining the two foci of the scan transfer device either lies substantially on a plane defined by the one-dimensional collimated light scan produced by the scan relay device or perpendicular to the plane defined by the one-dimensional collimated light scan produced by the scan relay device.

20. A method of scanning the retina of an eye as claimed in claim 14, wherein the method comprises the further step of providing a light detection device for detecting light reflected from the retina and using the light detection device to produce an image of the scanned area of the retina.

21. A method of scanning the retina of an eye as claimed in claim 14, wherein the method includes the further step of providing a wavefront sensing device for detecting wavefront aberration in the reflected light in the common optical path, and a wavefront compensation device including an adaptive optical element disposed in the common optical path between the source of collimated light and the eye, and using the wavefront compensation device to compensate for the wavefront aberration in the reflected light in the common optical path.

22. A method of scanning the retina of an eye as claimed in claim 14, wherein the method includes the further step of providing a program of predetermined selected operating parameters for the first and second scanning elements and following the program of predetermined selected operating parameters to produce a plurality of images of the retina.

23. A method of scanning the retina of an eye as claimed in claim 22, wherein the method includes the further step of combining at least a portion of the plurality of images of the retina to form a montage of the retina.

24. A method of scanning the retina of an eye as claimed in claim 14, wherein the method includes the further step of varying the amplitude of the angle of scan of the two-dimensional collimated light scan from the apparent point source.

25. A method of scanning the retina of an eye as claimed in claim 24, wherein the amplitude of the angle of scan of the two-dimensional collimated light scan from the apparent point source is varied by adjusting the magnification between the scanning elements and the scan transfer device and scan relay device.
